(19) United States
(12) Patent Application Publication
(10) Pub. No.: US 2012/0258502 A1
(43) Pub. Date: Oct. 11, 2012

(54) Method of producing recombinant plasmid dna using substantially solid growth medium

(76) Inventors: Vinod Pandiripally, Overland Park, KS (US); Kunal Bisariya, Overland Park, KS (US)

(21) Appl. No.: 13/506,252

(22) Filed: Apr. 6, 2012

Related U.S. Application Data
(60) Provisional application No. 61/516,795, filed on Apr. 8, 2011.

(51) Int. Cl. C12N 15/64

(52) U.S. Cl. 435/91.4

(57) ABSTRACT

A method of producing recombinant plasmid DNA using substantially solid growth medium and disposable vessels in place of conventional liquid fermentation processes. The method includes inoculating a host organism containing the recombinant plasmid DNA onto the substantially solid growth medium in a disposable vessel; allowing the host organism to grow on the growth medium under conditions conducive to such growth; removing the host organism from the growth medium and lysing the host organism to access the recombinant plasmid DNA; and purifying the recombinant plasmid DNA.

### RELATED APPLICATIONS

The present U.S. non-provisional patent application is related to and claims priority benefit of the U.S. provisional patent application titled METHOD OF PRODUCING RECOMBINANT PLASMID DNA, Ser. No. 61/516,795, filed Apr. 8, 2011. The identified prior-filed application is hereby incorporated by reference into the present application as though fully set forth herein.

### BACKGROUND OF THE INVENTION

1. Technical Field

The present invention broadly relates to methods for producing recombinant plasmid deoxyribonucleic acid (DNA).

2. Background

The field of recombinant plasmid DNA is rapidly expanding. Uses for this technology include non-viral gene therapeutics, DNA vaccines, and gene substitution vectors. Several recombinant plasmid DNA products are currently approved and licensed for veterinary applications, and several more are moving toward clinical trials as human vaccines. One obstacle standing in the way of commercializing this technology is the difficulty of efficiently and cost-effectively producing large amounts of the recombinant plasmid DNA, including problems with efficiently and cost-effectively generating a sufficient biomass of the host organism. Furthermore, different forms of recombinant plasmid DNA can require different production methods.

The prior art industrial production process 10, shown in FIG. 1, uses bio-reactors for large-scale liquid fermentation 12 in large culture volumes to generate a large amount of the host organism. Costly facilities and equipment are required to produce large amounts of product. Metabolic overload and stress is a common problem because the host cell is burdened with plasmid maintenance. Furthermore, the prior art fermentation process requires intense process development and tight control of process parameters such as carbon source concentration, temperature, pH, and dissolved oxygen. To increase product yields and ensure consistent product quality, key issues of conventional liquid fermentation process optimization and scale-up are aimed at maintaining optimum and homogenous reaction conditions, minimizing microbial stress exposure, and enhancing metabolic accuracy. For example, host cells close to the nutrient injection port are exposed to a high concentration of nutrients whereas cells at other locations are starved of substrate; the pressurized culture regime used to increase oxygen transfer may enhance the detrimental effect of carbon dioxide; and high cell density cultures, especially in large-scale fermentation, can generate detrimental levels of heat.

The problem of insufficient mixing at larger scales is aggravated by increasing vessel sizes: The opposing substrate and oxygen gradients along the vessel height, which are formed as a result of conventional fermenter design in which substrate feed usually occurs from the top and aeration usually occurs from the bottom, are more pronounced in larger reactors due to the longer distances to be covered. This leads to larger substrate and oxygen depletion zones, larger volumes of culture broth to be stirred, longer mixing times, and stronger hydraulic pressure gradients influencing the oxygen-transfer rate. In the case of glucose feeding, cells at the top of the fermenter are exposed to excess glucose concentrations and simultaneously suffer from oxygen limitations, whereas those at the bottom suffer from glucose starvation. Excess glucose concentrations result in overproduction of acetate, and the simultaneous deficiency of oxygen induces the formation of ethanol, hydrogen, formiate, lactate, and succinate.

Plasmid stability is the stable propagation of plasmids to daughter cells, and is an essential prerequisite for high product yields particularly in larger-scale production in which cultures pass a higher number of generations due to larger culture broth volumes and longer inoculation chains from the cell bank to the production stage. Plasmid stability is influenced by the plasmid properties as well as by process parameters like temperature, growth rates, and substrate concentrations. Thus, plasmid stability and plasmid numbers are adversely influenced, more difficult to control, and less easy to maintain in conventional larger-scale liquid fermentation.

Complex medium components are also a major source of process variability in liquid fermentation. For example, major constraints of high productivity recombinant bio-molecule expression during aerobic growth of Escherichia coli are the secretion of acetic acid, the effect of the medium, the effect of dissolved oxygen, and the lowering of the specific cellular protein yield.

Due to these and other problems and disadvantages in the prior art, a need exists for an improved method of producing recombinant plasmid DNA.

### SUMMARY OF THE INVENTION

The present invention addresses the above-discussed problems and disadvantages in the prior art to provide an improved method of producing recombinant plasmid DNA. Importantly, the present invention uses substantially solid growth medium and disposable vessels in place of conventional liquid fermentation processes, and provides a significant increase in recombinant plasmid DNA yield. More specifically, the present invention provides a method of large-scale production of recombinant plasmid DNA, wherein the method broadly comprises the steps of inoculating a host organism containing the recombinant plasmid DNA onto a substantially solid growth medium in a disposable vessel; allowing the host organism to grow on the growth medium under conditions conducive to such growth; removing the host organism from the growth medium and lysing the host organism to access the recombinant plasmid DNA; and purifying the recombinant plasmid DNA.

In various embodiments and implementations, the method may additionally or alternatively include one or more of the following features. The host organism may be a prokaryotic bacteria, such as Escherichia coli. The substantially solid nutrient medium may include one or more of sorbitol, sucrose, glucose, peptone, and yeast extract; one or more antibiotics; one or more trace elements or minerals for optimal growth of the host organism; or isopropyl-beta-D-thiogalactopyranoside at a concentration of less than approximately 25 micro-moles per milliliter. The host organism may be allowed to grow at a temperature of approximately between 15 degrees Celsius and 45 degrees Celsius. The recombinant plasmid DNA may contain a pUC temperature-inducible or chemical-inducible replication origin; a non-pUC replication origin; a viral or mammalian promoter DNA sequence; or a non-viral promoter DNA sequence. The recombinant plasmid DNA may also contain one or more consensus gene sequences that code for the production of immunogenic proteins or antigens; one or more targeted gene sequences for expression at required levels in the host organism; or one or more consensus targeted gene sequences for expressing immunogenic proteins or antigens at required levels in the host organism, leading to the production of antibodies or monoclonal antibodies. The step of purifying the recombinant plasmid DNA may be performed using column chromatography.

These and other features of the present invention are discussed in greater detail below.

### BRIEF DESCRIPTION OF THE DRAWINGS

The present invention is described herein with reference to the following drawing figures:

FIG. 1 (PRIOR ART) is a high-level flowchart of steps involved in a prior art method using liquid fermentation to produce recombinant plasmid DNA; and

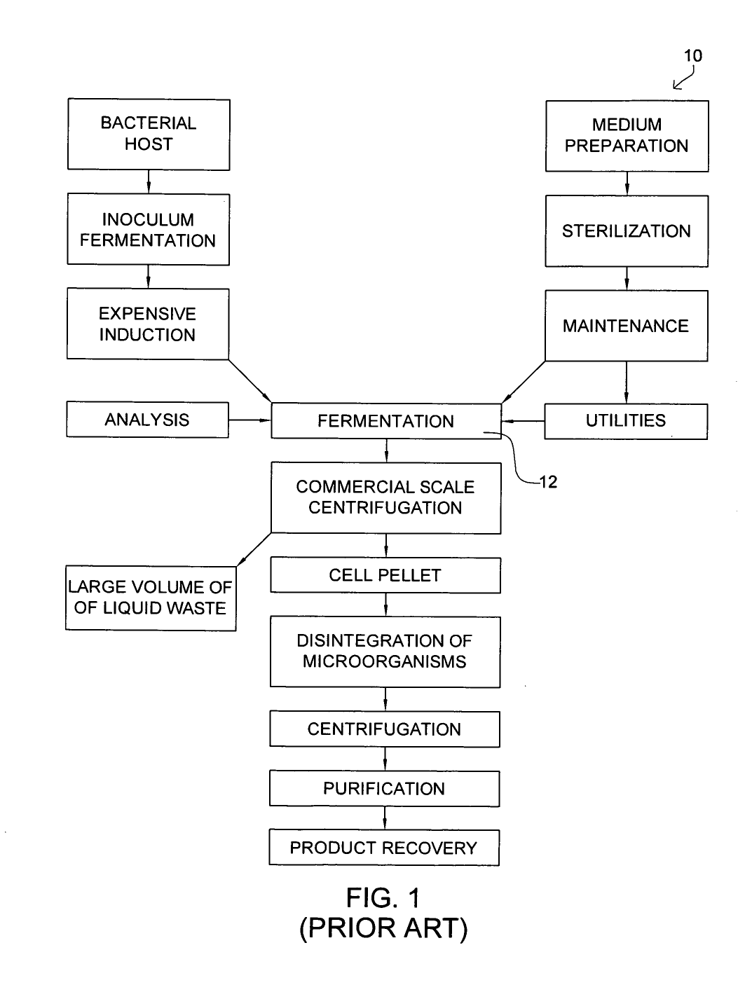

FIG. 2 is a high-level flowchart of steps involved in an embodiment of the method of the present invention for producing recombinant plasmid DNA.

### DETAILED DESCRIPTION OF THE INVENTION

The content of U.S. Pat. No. 7,229,792, issued Jun. 12, 2007, titled METHOD OF PRODUCING RECOMBINANT PROTEINS, is hereby incorporated by reference into the present application.

With reference to the various drawing figures, a method of producing recombinant plasmid DNA is herein described, shown, and otherwise disclosed in accordance with one or more preferred embodiments of the present invention. Broadly, the present invention concerns a method of producing recombinant plasmid DNA or shuttle plasmid DNA in a host organism grown on a substantially solid, i.e., solid or semi-solid, growth medium in a disposable vessel. The host organism may be, for example, a prokaryotic bacteria such as Escherichia coli, Pseudomonas fluorescens, or Corynebacterium glutamicum. The host cells are harvested from the substantially solid growth medium, and the plasmid DNA is recovered.

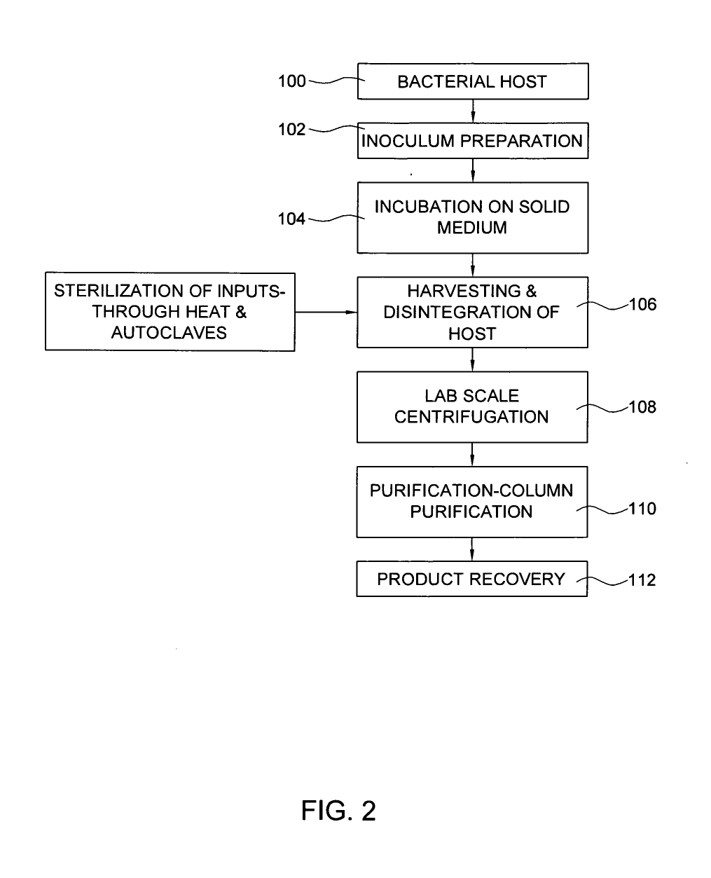

With reference to FIG. 2, in one embodiment the method of the present invention of producing recombinant plasmid DNA may proceed broadly as follows. In a first step 100, a recombinant host organism is created or otherwise obtained containing a recombinant, or “heterologous”, plasmid DNA including one or more targeted gene sequences to be expressed at sufficient levels. As necessary or desired, the recombinant plasmid DNA may contain one or more of the following: A pUC temperature-inducible or a chemical-inducible replication origin; a non-pUC replication origin; a viral promoter DNA sequence; a mammalian promoter DNA sequence; a non-viral promoter DNA sequence; or one or more consensus gene sequences that code for the production of immunogenic proteins or antigens.

In a second step 102, the host organism is inoculated onto the substantially solid growth medium in a disposable custom tray or other disposable vessel. The growth medium provides a source of necessary or desirable nutrients, antibiotics, and other components. For example, depending on the host organism, the growth medium may contain sorbitol, sucrose, glucose, peptone, and yeast extract; antibiotics such as kanamycin, ampicillin, or streptomycine; or essential trace elements/minerals such as selenium, nickel, or molybdenum to optimize the host organism's production of the recombinant plasmid DNA. The growth medium may also contain isopropyl-beta-D-thiogalactopyranoside at a concentration of less than approximately 25 micro-moles per milliliter. In a third step 104, the host organism is allowed to grow on the growth medium at an appropriate temperature and under any other necessary or desirable growth conditions. In a fourth step 106, the host organism is removed, or “harvested”, from the growth medium and lysed to access the contents. In a fifth step 108, the lysed host cells are centrifuged again. In a sixth step 110, the recombinant plasmid DNA is purified. In a seventh step 112, the purified recombinant plasmid DNA is recovered.

In one possible implementation of this method, in which the host organism is the prokaryotic bacteria E.coli strain DH5a, the above-described steps may proceed more specifically as follows. In the first step 100, the recombinant E.coli is created or otherwise obtained containing the recombinant plasmid DNA sequence that allows the one or more targeted gene sequences to be expressed at sufficient levels. In the second step 102, the host organism is inoculated onto the substantially solid growth medium containing nutrients such as carbon, nitrogen, minerals, and vitamins, in a disposable vessel. In the third step 104, the host organism is allowed to grow on the growth medium within a temperature range of approximately between 15 degrees C. and 45 degrees C.

In the fourth step 106, the host organism is harvested and lysed as follows. The organism is suspended in liquid and centrifuged at 10,000×g for 10 minutes to obtain a pellet of cells. The supernatant is removed and the tube is blotted upside-down on a paper towel to remove excess liquid. The cell pellet is re-suspended in an appropriate volume of cell re-suspension solution. Complete re-suspension can be important for obtaining optimal yields. An appropriate volume of alkaline cell lysis solution is added and mixed by inverting the tube and the resulting cell suspension. Host cells are lysed in NaOH/SDS buffer in the presence of RNase A. Phospholipid and protein components of the cell membrane are solubilized. As the cells are lysed, the cells' contents are released and the chromosomal and plasmid DNA and proteins are denatured.

When the lysis time is optimized, the recombinant plasmid DNA is released but the chromosomal DNA is not. In the fifth step 108, the lysed host cells are mixed with an appropriate volume of neutralization solution, and then centrifuged at 14,000×g for 30 minutes at 4 degrees C. The supernatant is decanted to a new tube while avoiding the precipitate. Alternatively, the cleared supernatant can be transferred by filter paper or an autoclaved coffee filter into a new centrifuge tube.

In the sixth step 110, the recombinant plasmid DNA is purified as follows. The filtered lysate is applied to an appropriate chromatography column and allowed to enter the resin by gravity flow. The column is washed with an appropriate volume of wash solution, and the wash solution is allowed to move through the column by gravity flow. The first half of the volume of wash solution should be sufficient to remove all contaminants in the majority of recombinant plasmid DNA preparations. The second half may be necessary when dealing with large volumes producing large amounts of carbohydrates. The plasmid is eluted with an appropriate volume of elution solution or sterile pure water. The plasmid DNA is precipitated by adding an appropriate volume of room-temperature isopropanol to the eluted DNA.

In the seventh step 112, the purified recombinant plasmid DNA is recovered as follows. The result of the preceding step is mixed and centrifuged at approximately 15,000×g for 30 minutes at 4 degrees C. The supernatant is carefully removed. The plasmid DNA pellet is washed with an appropriate volume of endotoxin-free room-temperature 70% ethanol and centrifuged at approximately 15,000×g for 30 minutes. The supernatant is removed without disturbing the pellet. The pellet is air-dried for 15 minutes, and the plasmid DNA is re-dissolved in an appropriate volume of endotoxin-free solution.

To illustrate the efficiency of the method of the present invention, two different recombinant plasmid DNA products were produced using disposable multi-liter solid medium vessels. After approximately 16 to 18 hours of bacterial growth, the average yield of biomass (gram wet biomass weight/liter of culture medium) was approximately 40 grams per liter and the corresponding average mean specific plasmid DNA yield (mg of plasmid/g wet weight) was approximately 8 micrograms of plasmid DNA per gram of wet biomass weight. This high specific yield significantly reduces the burden on the plasmid DNA production processes.

By way of comparison, whereas a conventional fermentation process produces a biomass yield of 23 grams per liter and a specific yield of 0.7 micrograms per gram, and whereas a conventional wave bioreactor process produces a biomass yield of 27 grams per liter and a specific yield of 1 microgram per gram, the method of the present invention produces a biomass of 40 grams per liter and a specific yield of 8 micrograms per gram (wherein specific yield values have been normalized to represent the micrograms of plasmid obtained per gram of harvested bacteria).

Thus, increased plasmid DNA production occurs along with host cell growth on the substantially solid growth medium using a disposable vessel. This addresses problems associated with prior art processes that are dependent on liquid-medium fermentation. Unlike prior art processes, the method of the present invention provides constitutive high plasmid production throughout the host's growth phase. Maintaining increased plasmid DNA production during the biomass accumulation creates an environment that is adverse to plasmid-free host cells. Thus, maintaining constant conditions, such as temperature, pH, and composition of the growth medium, throughout the process is desirable and leads to increased plasmid yields while preserving plasmid quality.

Other benefits of the method of the present invention include: The method reduces or eliminates expensive and unreliable equipment, which minimizes overall capital investment; it provides total product isolation in a continuous flow-path; it provides gentle but robust processing which results in higher specific yield ratios of plasmid DNA to host organism biomass; it is easily scalable from research and development levels to production levels; it provides improved batch control; it reduces or eliminates toxic chemicals, uses less water, and produces fewer waste products; and it uses disposable vessels which eliminates labor costs associated with cleaning, eliminates the potential for product cross-contamination, and allows for quicker turn-around times and multiple production runs in a day.

Although the invention has been disclosed with reference to various particular embodiments, it is understood that equivalents may be employed and substitutions made herein without departing from the contemplated scope of the invention.

1. A method of large-scale production of recombinant plasmid DNA, the method comprising the steps of:
inoculating a host organism containing the recombinant plasmid DNA onto a substantially solid growth medium in a disposable vessel;
allowing the host organism to grow on the growth medium under conditions conducive to such growth;
removing the host organism from the growth medium and lysing the host organism to access the recombinant plasmid DNA; and
purifying the recombinant plasmid DNA.

2. The method as set forth in claim 1, wherein the host organism is a prokaryotic bacteria.

3. The method as set forth in claim 2, wherein the host organism is Escherichia coli.

4. The method as set forth in claim 1, wherein the substantially solid nutrient medium includes one or more of sorbitol, sucrose, glucose, peptone, and yeast extract.

5. The method as set forth in claim 1, wherein the substantially solid nutrient medium includes one or more antibiotics.

6. The method as set forth in claim 1, wherein the substantially solid nutrient medium includes one or more trace elements for optimal growth of the host organism.

7. The method as set forth in claim 1, wherein the substantially solid nutrient growth medium includes isopropyl-beta-D-thiogalactopyranoside at a concentration of less than approximately 25 micro-moles per milliliter.

8. The method as set forth in claim 1, wherein the host organism is allowed to grow at a temperature of approximately between 15 degrees Celsius and 45 degrees Celsius.

9. The method as set forth in claim 1, wherein the recombinant plasmid DNA contains a pUC temperature-inducible replication origin.

10. The method as set forth in claim 1, wherein the recombinant plasmid DNA contains a pUC chemical-inducible replication origin.

11. The method as set forth in claim 1, wherein the recombinant plasmid DNA contains a non-pUC replication origin.

12. The method as set forth in claim 1, wherein the recombinant plasmid DNA contains a viral promoter DNA sequence.

13. The method as set forth in claim 1, wherein the recombinant plasmid DNA contains a mammalian promoter DNA sequence.

14. The method as set forth in claim 1, wherein the recombinant plasmid DNA contains a non-viral promoter DNA sequence.

15. The method as set forth in claim 1, wherein the recombinant plasmid DNA contains one or more consensus gene sequences that code for the production of immunogenic proteins or antigens.

16. The method as set forth in claim 15, wherein the one or more consensus targeted gene sequences are for expressing immunogenic protein or antigens at a desired level in the host organism, leading to the production of antibodies or monoclonal antibodies.

17. The method as set forth in claim 1, wherein the recombinant plasmid DNA contains one or more targeted gene sequences for expression at a desired level in the host organism.

18. The method as set forth in claim 1, wherein the step of purifying the recombinant plasmid DNA is performed using column chromatography.

19. A method of large-scale production of recombinant plasmid DNA including a desired gene sequence, the method comprising the steps of:
obtaining a host organism containing the recombinant plasmid DNA including the desired gene sequence, wherein the host organism is a prokaryotic bacteria;
inoculating the host organism onto a substantially solid growth medium in a disposable vessel;
allowing the host organism to grow on the growth medium under conditions conducive to such growth;
removing the host organism from the growth medium and lysing the host organism to access the recombinant plasmid DNA; and
purifying the recombinant plasmid DNA.

20. A method of large-scale production of recombinant plasmid DNA including a desired gene sequence, the method comprising the steps of:
obtaining a host organism containing the recombinant plasmid DNA including the desired gene sequence, wherein the host organism is a prokaryotic bacteria and the recombinant plasmid DNA contains an inducible replication origin and a promoter DNA sequence;
inoculating the host organism onto a substantially solid growth medium in a disposable vessel;
allowing the host organism to grow on the growth medium under conditions conducive to such growth;
removing the host organism from the growth medium and lysing the host organism to access the recombinant plasmid DNA; and
purifying the recombinant plasmid DNA.
